(19) United States
(12) Patent Application Publication
(10) Pub. No.: US 2004/0166569 A1
(43) Pub. Date: Aug. 26, 2004

(54) Thermal cycling methods and apparatus

(76) Inventors: Andre Marziali, North Vancouver (CA); Shu Gee Chow, Vancouver (CA)

Correspondence Address: PIPER RUDNICK, P. O. BOX 64807, CHICAGO, IL, 60664-0807, US

(21) Appl. No.: 10/483,800

(22) PCT Filed: Jul. 12, 2002
(86) PCT No.: PCT/CA02/01075

(51) Int. Cl. C12M 1/38

(52) U.S. Cl. 435/91.2; 435/287.2; 435/303.1; 435/286.1

(57) ABSTRACT

A thermal cycling method maintains inner wall surfaces of a well at temperatures greater than a temperature of a liquid being subjected to thermal cycling. The method may be applied in performing the polymerase chain reaction (PCR). Apparatus for performing thermal cycling provides one or more wells having regions of reduced thermal conductivity.

### CROSS REFERENCE TO RELATED APPLICATIONS

This application claims the benefit of the filing date of U.S. patent application No. 60/304,781 filed on 13 Jul. 2001.

### TECHNICAL FIELD

This invention relates to thermal cycling of liquid volumes for the purpose of promoting chemical reactions. The invention may be applied to promoting the polymerase chain reaction (PCR). Specific embodiments of the invention provide methods for performing thermal cycling of small volumes of liquid and apparatus for performing thermal cycling of small volumes of liquid. Specific embodiments of the invention include multi-well plates for thermal cycling of biological samples to perform the duplication of nucleic acid sequences by mechanisms such as PCR.

### BACKGROUND

Some facets of biological research involve the duplication of nucleic acid sequences. A sample of biological material including one or more nucleic acid sequences can be exposed to conditions, which promote a reaction that duplicates those nucleic acid sequences. The conditions for promoting such reactions often involve thermal cycling of the sample in the presence of appropriate reagents. Various techniques for performing thermal cycling of biological samples are well known.

Because it is often desirable to test a large number of biological samples at the same time, and under similar conditions, it is common to provide multi-well plates. Such plates have a number of wells, each of which is capable of holding a small volume of a biological sample together with suitable reagents. Typically each well in such a multi-well plate holds 3 μl or more of sample and reagents. The number of wells in a plate is variable. Some standard thermal cycling apparatus have plates with 384 wells, while other standard plates have 96 wells.

Multi-well plates are typically mounted in an apparatus which places each well in good thermal contact with a temperature-controlled block. A temperature controller controls a suitable heating/cooling system associated with the block. The apparatus normally provides a lid to close off the wells. The lid is typically heated to a temperature of slightly higher than 100° C. For example, the lid may be maintained at a temperature in the range of 100° C. to 103° C.

Using a multi-well plate apparatus, many profiles of temperature as a function of time (“temperature-time profiles”) are possible. During a typical thermal cycling process used to promote PCR, a sample is repetitively heated to a temperature of approximately 95° C. and cooled to a temperature near 50° C.

The reagents used to promote reactions such as the polymerase chain reaction can be very expensive. Biological samples themselves may be scarce and may only be available in very small quantities. It would be desirable to be able to practice thermal cycling with smaller volumes of samples and reagents. However, it is not practical to use sample sizes below approximately 3 μl in currently-available thermal cycling apparatus, because physical effects that occur between the apparatus and the sample tend to interfere with reactions.

There is a need for methods and apparatus which permit the use of smaller sample and reagent volumes in thermal cycling. As there is a relatively large installed base of thermal cycling equipment, there is a particular need for such methods and apparatus suitable for use with currently available thermal cycling equipment.

### SUMMARY OF THE INVENTION

This invention provides a method for thermal cycling of a liquid sample. The method comprises: placing a volume of the liquid sample into a well and varying the temperature of the liquid sample according to a desired temperature-time profile. While varying the temperature of the liquid sample, the method maintains a temperature of one or more regions on an inner surface of the well at temperatures at least 1½° C. greater than the temperature of the liquid sample. The one or more regions maintained at higher temperatures constitute 50% or more of an area of the inner surface of the well above a separation level.

The separation level may be one of: a level of the liquid sample; a level between a lower 3 μl of the well and a part of the well above the lower 3 μl of the well; where the well has a volume of 6 μl or less, a level separating upper and lower halves of the well's volume, and a level of a known sample volume at a standard temperature.

Varying the temperature of the liquid sample may comprise cycling the liquid sample between a number of temperatures. PCR protocols in which temperature is cycled between three temperatures are common. Two temperature PCR protocols are also used. In PCR, each of the temperatures may be in the range of 0° C. to 100° C. The lowest temperatures used are typically in the range of 40° C. to 60° C. and the highest temperatures are typically in the range of 92° C. to 98° C.

The one or more regions on the inner surface of the wall may constitute 75 percent or more of the area of the inner surface of the well above the separation level.

Varying the temperature of the liquid sample may involve placing the well in good thermal contact with a temperature-controlled block and varying a temperature of the temperature-controlled block. In some embodiments, maintaining a temperature of one or more regions on an inner surface of the well at temperatures at least 1½° C. greater than the temperature of the liquid sample may involve placing the regions in good thermal contact with a temperature-controlled plate, body of gas or body of liquid and controlling a temperature of the temperature-controlled plate, body of gas or body of liquid.

The volume of the liquid sample may be less than 3 μl and, in some embodiments is, less than 1 μl.

Another aspect of the invention provides an apparatus for performing thermal cycling on a volume of a liquid. The apparatus comprises a well with a wall having an inner surface surrounding a bore. The well has a sample holding volume located in the bore at a lower end of the well. The apparatus also comprises a block with a socket for receiving the well and a temperature controller for controlling a temperature of the block. The apparatus also comprises a heated lid capable of being brought into good thermal contact with an upper end of the well. When the well is received in the socket, the sample-holding volume has a first thermal contact with the block. One or more regions on the inner surface, which constitute 50 percent or more of an area of the inner surface of the well above the sample-holding volume, have a second thermal contact with the block. The first thermal contact is closer than the second thermal contact.

When the well is received in the socket, the lower end of the well may be touching the block and there may be an air gap between the well and portions of the block above the sample-holding region.

An upper portion of the well may comprise a layer of a material which is thermally insulating relative to a material of the lower end of the well.

The well may comprise a region of reduced thermal conductivity between the one or more regions on the inner surface and the lower end of the well. The region of reduced thermal conductivity may extend circumferentially around the wall of the well. The region of reduced thermal conductivity may comprise a region within which a thickness of the wall is reduced. The region of reduced thermal conductivity may comprise a region within which the wall is made of a material having a reduced thermal conductivity in comparison to a material of the wall adjacent the sample-holding volume.

The sample-holding volume may have a cross-sectional area smaller than a cross-sectional area of the bore above the sample-holding volume.

The one or more regions on the inner surface may have thermal proximities to the block of 19 or less. The one or more regions on the inner surface may have thermal proximities to the heated lid of 1/19 or greater.

The block may comprise an array of sockets and the apparatus may comprise a plurality of wells connected together and engageable in corresponding ones of the sockets.

The sample-holding volume may be less than 3 μl and in some embodiments, is less than 1 μl.

Another aspect of the invention comprises a well for use in conjunction with a thermal cycling apparatus having a heated lid and a temperature-controlled block. The temperature controlled block has a socket for receiving the well to expose a volume of liquid to thermal cycling. The well comprises: a wall having an inner surface surrounding a bore and a sample-holding volume located in the bore at a lower end of the well. There are one or more regions, which constitute 50% or more of an area of the inner surface of the well above the sample-holding volume. When the well is received in the socket, these regions have thermal proximities of 1/19 or greater to the heated lid and thermal proximities of 19 or less to the block.

Another aspect of the invention provides an apparatus for performing thermal cycling on a liquid sample. The apparatus comprises: a well having a wall surrounding a bore; a block having a socket for receiving the well and a temperature controller for controlling the temperature of the block; and a heated lid capable of being brought into good thermal contact with an upper end of the well. When the well is received in the socket, the well comprises a lower region, which has a first thermal contact with the block, and an upper region comprising portions that constitute 50% or more of an area of an inner surface of the wall, which have a second thermal contact with the block. The first thermal contact is closer than the second thermal contact.

A separation level between the lower region and the upper region may be one of: a level of the liquid sample; a level between a lower 3 μl of the well and a part of the well above the lower 3 μl of the well; especially where the well has a volume of 6 μl or less, a level separating upper and lower halves of the well's volume, and a level of a known sample volume at a standard temperature.

Further features of the invention and specific embodiments of the invention are described below.

### BRIEF DESCRIPTION OF DRAWINGS

In drawings which illustrate non-limiting embodiments of the invention:

FIG. 1 is a cross-sectional view through a portion of a prior art thermal cycling apparatus;

FIG. 2 is a typical plot of temperature versus time for a thermal cycling process;

FIG. 3 is a schematic illustration of thermal contact between portions of a well;

FIG. 4 is a cross-sectional view through a well in a thermal cycling apparatus according to one embodiment of the invention;

FIG. 5 is a cross-sectional view through the well of FIG. 4 with superposed isotherms;

FIG. 6 is a cross-sectional view through a well in a thermal cycling apparatus according to an alternative embodiment of the invention;

FIG. 7 is an illustrative schematic model of a well according to a further alternative embodiment of this invention;

FIG. 8 is a block diagram illustrating a method according to the invention; and,

FIGS. 9A and 9B are sections through an upper end of a well in embodiments of the invention wherein the well is sealed with a plug which projects downwardly into the well.

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

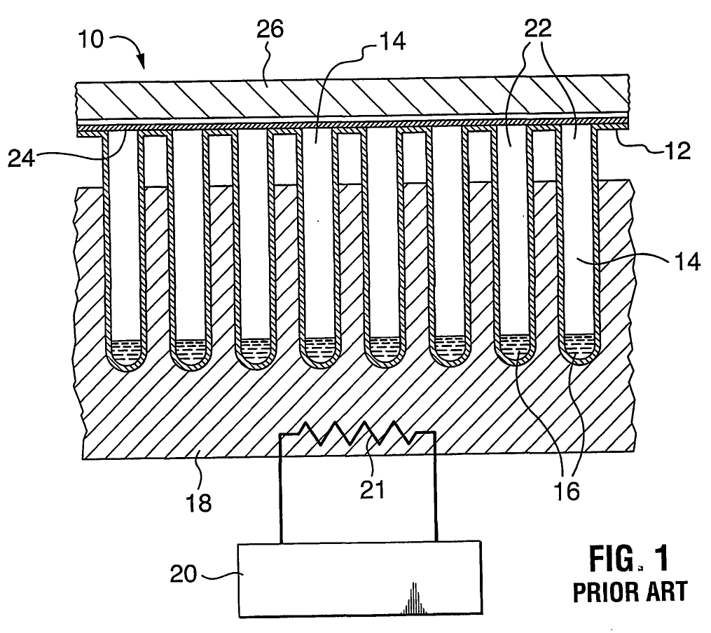

FIG. 1 shows a cross-sectional view through a typical prior art thermal cycling apparatus 10. Apparatus 10 includes a plate 12 in which a number of wells 14 are formed. One common type of prior art thermal cycling apparatus has 384 wells 14 on each plate 12. Each well 14 contains a volume, which typically exceeds 3 μl, of liquid 16. Liquid 16 may comprise, for example, a biological sample, a solvent and reagents. The reagents contained in liquid 16 may include enzymes that promote PCR. In general, however, liquid 16 may comprise any number of reactants to be subjected to a thermal cycling process.

Each well 14 is in good thermal contact with a temperature-controlled block 18. Temperature controller 20 controls a heating/cooling system 21 to cause a temperature of temperature-controlled block 18 to follow a desired temperature-time profile. The openings 22 of wells 14 are each closed off by an adhesive sheet 24, which covers openings 22. A hot lid 26 is provided on top of adhesive sheet 24.

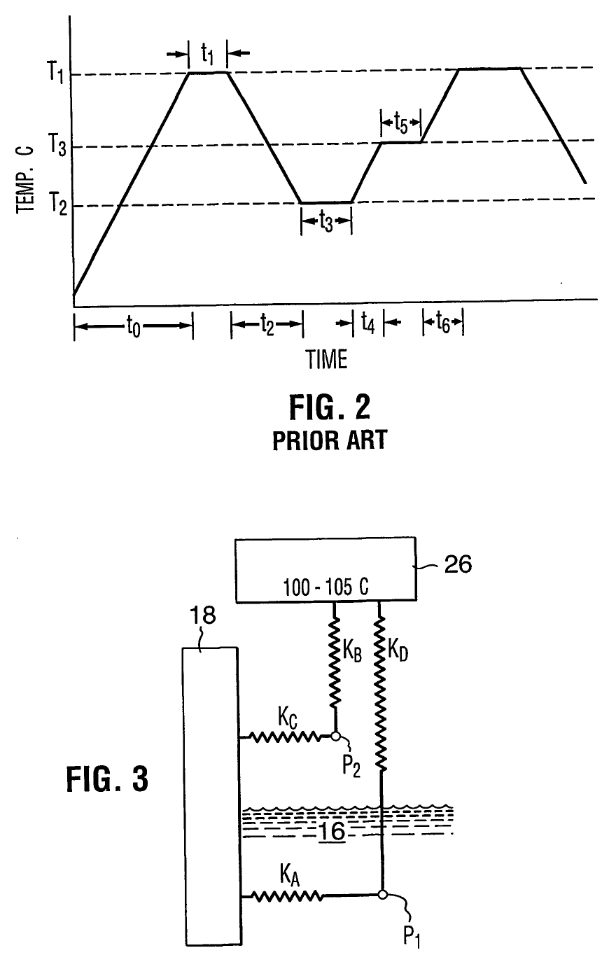

FIG. 2 depicts a graph of a portion of a temperature-time profile for a typical thermal cycling process. Initially, the illustrated cycling process involve heating liquid 16 from room temperature to a temperature T₁ during time t₀. Following the initial ramp up from room temperature during time t₀, the process involves a number of cycles. The first cycle includes holding liquid 16 at a temperature T₁ during a time t₁, cooling liquid 16 to a temperature T₂ during time t₂, holding liquid 16 at a temperature T₂ for a time t₃, heating liquid 16 to an intermediate temperature T₃ during time t₄, holding liquid 16 at temperature T₃ for a time t₅, and then reheating liquid 16 to temperature T₁, during time t₆. For PCR applications, T₁, may be 95° C., T₂ may be 50° C. and T₃ may be 70° C. In other applications, T₁, T₂ and T₃ may be other, different, temperatures. A temperature-time profile may also involve cycles comprising two, or more than three distinct temperatures.

For PCR or similar processes that involve the thermal cycling of small volumes of liquid 16, the inventors have determined that the loss of liquid 16 during the thermal cycling process is a significant problem. One mechanism by which a considerable amount of liquid 16 is lost involves evaporation and condensation. During warmer parts of a thermal cycle (for example, during time t₁ and possibly, at the earlier stages of time t₂ or at the latter stages of time t₆) a portion of liquid 16 evaporates. When the temperature of the walls of well 14 falls during a cooling portion of the cycle (for example, t₂), some of the evaporated liquid condenses onto the walls of well 14. Some of the evaporated liquid that condenses on the walls of well 14 adheres to the walls and does not rejoin the bulk of liquid 16. As the remaining volume of liquid 16 is reduced by this evaporation and condensation process, the proportion of the remaining volume of liquid 16 that is adhering to the walls increases. Using conventional thermal cycling equipment, one cannot effectively perform thermal cycling on volumes of liquid 16 which are smaller than a certain limit (typically in the range of 3 μl). At volumes below this limit, the change in concentration of the reagents contained in liquid 16 (caused by evaporative losses of liquid 16) may adversely affect the reactions taking place.

This invention provides apparatus and methods for thermal cycling. The apparatus includes one or more wells for holding liquids 16, which may comprise, for example, biological samples, solvents and/or reagents. Liquid 16 may include enzymes that promote PCR. In general, however, liquid 16 may comprise any liquid to be subjected to a thermal cycling process. The apparatus maintains at least two temperature zones on the wall of each well at least during portions of the thermal cycling process in which liquid 16 is being cooled. For at least one portion of the well wall located above a separation level, the apparatus maintains a temperature on the inner surface of the well wall somewhat higher than a temperature of the liquid 16. In contrast, portions of the well wall located below the separation level are maintained at substantially the same temperature as liquid 16. The separation level is a level between a volume within the well which is intended to hold liquid sample 16 and a volume of the well which is above liquid 16. The separation level may be any of:

the level of a surface of liquid 16,

a level between a lower 3 μl volume of well 14 and a part of the well above the lower 3 μl volume;

in a case where the well has a volume of 6 μl or less, a level separating upper and lower halves of the well's volume,

a step in a diameter of well 14 which demarcates an upper edge of a sample-holding volume below the step; and,

a level of a known sample volume at a standard temperature.

The inventors have determined that this multi-zone temperature profile tends to reduce the amount of liquid lost to condensation of vapors from liquid 16 onto the well wall, which, in turn, makes it practical to perform thermal cycling processes using smaller volumes of liquid 16.

One aspect of this invention provides a thermal cycling apparatus capable of maintaining a multi-zone temperature profile on the wall of a well. A well (and typically a plurality of wells) is constructed so that the thermal conductivity of its wall varies in different regions.

FIG. 3 is a schematic illustration which depicts thermal contact between portions of a well made in accordance with one embodiment of the invention. A well (not shown in FIG. 3) may be part of a multi-well plate. The plate may be in thermal cycling apparatus which includes a temperature-controlled block 18 and a heated lid 26. P₁ and P₂ represent points on the inner wall of the well. Point P₁ is in a lower region of the well wall, below the separation level. Point P₂ is in an upper region of the well wall, above the separation level. Thermal contact between any two elements of FIG. 3 is schematically illustrated by zig-zag lines. “Thermal contact” between two elements means the sum of thermal conductivities over all paths connecting the two elements. In FIG. 3, the thermal contact between point P₁ and block 18 is represented by K_{A}, thermal contact between point P₂ and heated lid 26 is represented by K_{B}, thermal contact between point P₂ and block 18 is represented by K_{C}, and thermal contact between point P₁ and heated lid 26 is represented by K_{D}.

When two elements at different temperatures are in thermal contact with one another, they will eventually reach an equilibrium temperature distribution. With better thermal contact between the two elements, the time taken to reach the equilibrium temperature distribution is decreased.

In wells according to some embodiments of this invention, points P₂ are in significantly closer thermal contact with heated lid 26 than are points P₁. In situations where heated lid 26 is at a higher temperature than temperature-controlled block 18, this difference in thermal contact results in points P₂ having greater temperatures than points P₁.

In general, it is desirable for the temperature of point P₁ to track the temperature of temperature-controlled block 18 within X° C. (for example, X may be 1° C.). To achieve this, the following relationship should hold:

$$\frac{1}{1 + \frac{{K}_{A}}{{K}_{D}}} < \frac{X}{Δ T} \qquad ( 1 )$$

where ΔT is the largest temperature differential between heated lid 26 and block 18 during which the temperature at point P₁ should be maintained within X° C. of block 18. In some typical applications, X≈50. For example, if X=1 and ΔT=50 then K_{A}/K_{D}>49.

It is also desirable that point P₂ be warmer than point P₁ by Y° C. or more (for example, Y may be 1½° C.). To achieve this, the following relationship should hold:

$$X + Y < \frac{Δ T}{1 + \frac{{K}_{C}}{{K}_{B}}} \qquad ( 2 )$$

For example, if X=1, Y=1½ and ΔT=50 then K_{C}/K_{B}<19.

The “relative thermal proximity” of a point to heated lid 26 relative to temperature-controlled block 18 is used herein to mean the ratio of the thermal conductivity K_{LID} between the point and heated lid 26 to the thermal conductivity K_{BLOCK} between the point and block 18. The relative thermal proximity of the point to block 18 relative to heated lid 16 is the ratio K_{BLOCK}/K_{LID}. The thermal proximity of the point to heated lid 26 can, in the alternative, be expressed as a percentage of the total heat flow to or from the point which flows between the point and heated lid 26 under circumstances where heated lid 26 and temperature-controlled block 18 are both maintained at the same first temperature and the point in question is maintained at a second temperature which is different from, but within 50° C. of, the first temperature. Thermal proximity expressed in this second way is different from the relative thermal proximity and is called the “percentage thermal proximity” herein. The relative thermal proximity and percentage thermal proximity of a point on an inner surface of a well to heated lid 26 (or to temperature-controlled block 18) may be determined by performing finite element analysis on the well.

One aspect of the invention provides for a well constructed so that the inner surface of its wall has a region (or possibly a plurality of component regions), which occupies at least 50% of the inner surface area of the wall above the separation level. The region on the inner surface of the wall above the separation level has a thermal proximity

$$\frac{{K}_{LID}}{{K}_{BLOCK}}$$

to temperature-controlled block 18 of 19 or less or a thermal proximity

$$\frac{{K}_{BLOCK}}{{K}_{LID}}$$

to heated lid 26 of 1/19 or greater. In some embodiments the percentage thermal proximity of block 18 is 80% or less and the percentage thermal proximity of such points to heated lid 26 is 20% or greater.

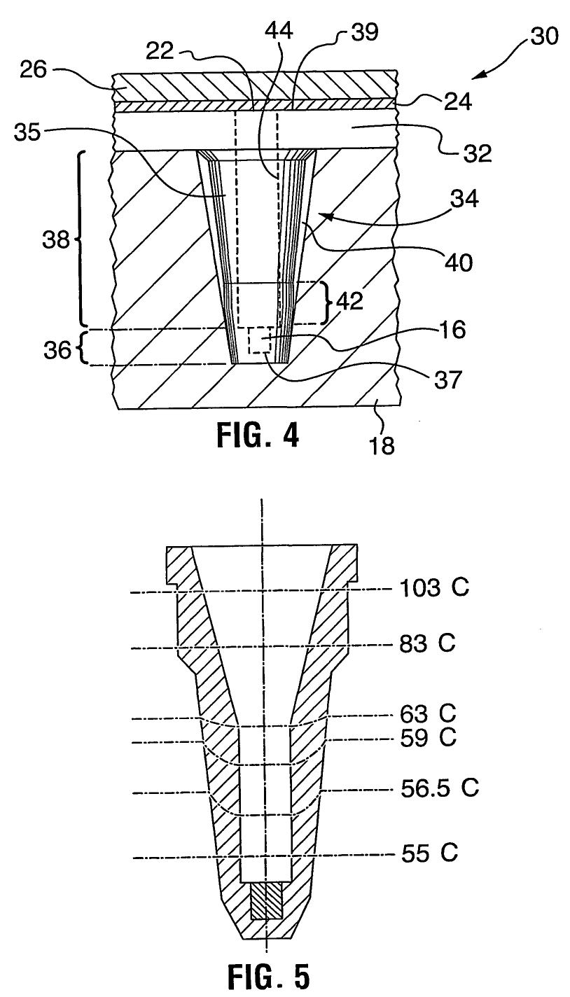

FIG. 4 illustrates an apparatus 30 according to one embodiment of the invention. Typically, although not necessarily, apparatus 30 comprises a plurality of wells 34, only one of which is depicted in FIG. 4. In the illustrated embodiment, apparatus 30 includes a plate 32 which supports a plurality of wells 34. Each well 34 has a wall 35 and is capable of receiving a volume of liquid 16 to be subjected to thermal cycling. The material of wall 35 may be a plastic, such as polypropylene, or may be another suitable material. The inner surfaces of well 34 may be treated to prevent inactivation of polymerase enzymes in any suitable manner, including the application of surface treatments known to those skilled in the art.

Well 34 comprises a lower region 36 below a separation level 37 which, in this case, corresponds to a surface level of liquid 16. In lower region 36, the material of wall 35 is in good thermal contact with temperature-controlled block 18. Well 34 also includes an upper region 38 in which there is an air space 40 separating the material of wall 35 from temperature-controlled block 18. The upper end 39 of well 34 is in thermal contact with heated lid 26. Opening 22 of well 34 is closed by a suitable closure, such as a plug or a layer of adhesive film 24.

In each well 34 of the illustrated embodiment, there is a region 42. Region 42 has a relatively low thermal conductivity. There is reduced thermal contact between points on well 34 above and below region 42. Region 42 is located generally at a lower end of upper region 38 and extends circumferentially around wall 35. The relatively low thermal contact between points above region 42 and points below region 42 may be achieved in a number of ways including, without limitation, by:

making wall 35 thin in the vicinity of region 42;

making a portion of wall 35 in region 42 from a material having a lower thermal conductivity than the material from which other parts of wall 35 are made. The lower thermal conductivity material may be completely different from the material in other parts of wall 35 or may comprise the same or a similar material mixed with another material which decreases its thermal conductivity; and/or

enhancing the thermal conductivity of wall 35 above and/or below region 42 by, for example, applying a layer of a high thermal conductivity material, such as a metal, to wall 35.

In the embodiment of FIG. 4, well 34 includes a lowermost sample-holding volume 37, which holds liquid 16. Volume 37 is capable of holding a liquid sample of up to a given size. In general, the size of sample-holding volume 37 depends on the particular application. In preferred embodiments, sample-holding volume 37 is sized to hold liquid volumes 16 which are 3 μl or less. Sample-holding volume 37 may be dimensioned to hold less than 3 μl of fluid 16 or even less than 1 μl of fluid 16. In the illustrated embodiment, volume 37 has a smaller horizontal cross-sectional area than other higher up portions of well 34. This provides a relatively small horizontal surface area at the surface of liquid 16. In some embodiments, changes in internal diameter of well 34 occur smoothly so that there are no steps inside well 34 which would catch on pipettor needles being inserted into the well 34.

In lower region 36, wall 35 is in good thermal contact with block 18. As a result, wall 35 in lower region 36 and liquid 16 are maintained at roughly the same temperature as block 18. However, in use, heated lid 26 may be maintained at a temperature greater than that of temperature-controlled block 18. For example, in some PCR applications, heated lid 26 is maintained at a temperature in the range of 100° C. to 105° C. Heat flowing from heated lid 26 to wall 35 in upper region 38 maintains the inner surface of wall 35 in upper region 38 at a temperature greater than that of liquid 16. Points on the inner surface of wall 35 in upper region 38 may be at the temperature of heated lid 26 or at temperatures intermediate the temperatures of heated lid 26 and liquid 16. In some embodiments of the invention, in at least 50% of the inner surface area of wall 35 in upper region 38, the temperature is maintained at least 1½° C. greater than that of liquid 16 and preferably at least 2° C. greater than that of liquid 16, while the temperature of liquid 16 is cycled. The portions of the inner surface of wall 35 in which this temperature differential exists may be located in one or more sub-regions of wall 35 within upper region 38. As noted above, thermal cycling is performed between temperatures in the range of 0° C. to 100° C., and most typically in the range of 40° C. to 98° C.

FIG. 5 shows the temperatures within the wall 35 of well 34 of FIG. 4 when heated lid 26 is maintained at a temperature of 103 IC and temperature-controlled block 18 is held at a temperature of 55° C. It can be seen that the inner surface of wall 35 in upper region 38 remains warmer than the inner surface of wall 35 in lower region 36. Because of this multi-zone temperature profile, condensation of evaporated liquids tends to occur preferentially into lower region 36. As shown in FIG. 5, the well of the invention causes the temperature profile of the inner surface of the well to exhibit a stepwise increase at a level which is near the surface of the liquid in the sample-holding volume at the bottom of the well. This temperature profile is characterized by a fairly constant temperature in parts of the inner wall which define the sample-holding volume and a sharp increase in temperature at a location near the upper edge of the sample-holding volume.

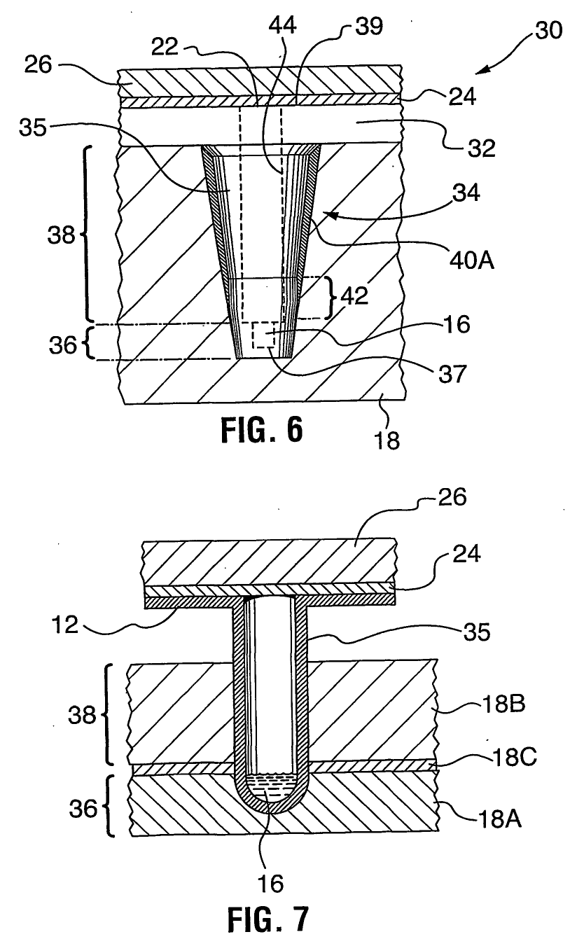

FIG. 6 illustrates an alternative embodiment of the invention where, instead of an air space 40 surrounding well 34, there is a layer 40A of a different material. The material of layer 40A has a lower thermal conductivity than the material of wall 35. Layer 40A extends circumferentially around wall 35 between the inner surface of wall 35 and block 18.

FIG. 7 shows a further alternative embodiment of the invention wherein the temperature-controlled block comprises a first portion 18A, a second portion 18B and a thermally insulating layer 18C that separates portions 18A and 18B. Both liquid 16 and lower region 36 of wall 35 are in close thermal contact with the first portion 18A of the block. Upper region 38 of wall 35 is in close thermal contact with the second portion 18B of the block. In operation, portion 18B of the temperature-controlled block is maintained at a temperature slightly higher than region 18A. For example, portion 18B might be maintained at a temperature exceeding that of portion 18A by 1° C. or more, and preferably by 2° C. or more. In a further alternative implementation of the invention, portion 18B is replaced with a region containing a temperature-controlled liquid or gas.

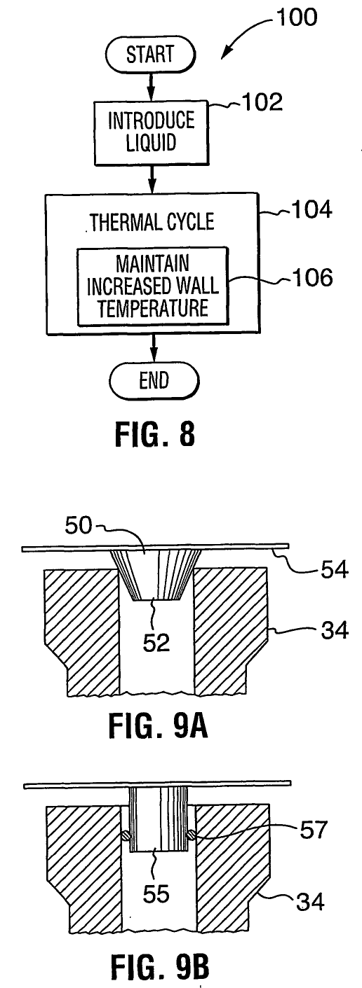

FIG. 8 illustrates a method 100 according to the invention. Method 100 begins by introducing a liquid sample into a well (block 102). In block 104, a temperature of the liquid is varied according to a desired temperature-time profile. While varying the temperature of the liquid, the temperatures of one or more regions on an inner surface of the well are maintained at least 1½° C. greater than that of the liquid as indicated by block 106. Preferably, the one or more regions constitute 50 percent or more of the area of the inner surface of the well that is above the separation level.

FIGS. 9A and 9B show embodiments of the invention in which sealing plugs 50 are provided to reduce the escape of fluid vapors from wells 34. Sealing plugs 50 extend into the bores of wells 34. In the embodiment of FIG. 9A, sealing plugs 50 comprise truncated-conical studs 52 which protrude from a plate 54. In the embodiment of FIG. 9B, sealing plugs 50 comprise generally cylindrical studs 55 which extend into the bores of wells 34. Studs 55 comprise o-rings 57 which seal against the inner wall of well 34.

### EXAMPLE

A number of wells according to this invention were prepared. Some were made from sections of heat-shrinkable Teflon™ tubing, others were made from injection-molded polyethylene. The construction of each well is as shown in FIG. 3. Liquid samples of 500 nl and 600 nl were loaded into each of four prototype wells. The upper ends of the wells were sealed with a self-adhesive film. Some of the wells were exposed to 25 cycles of thermal cycling, wherein each cycle involved holding the liquid at 96° C. for 10 seconds followed by holding the liquid at 50° C. for 5 seconds. Other wells were cycled using the more demanding “dye terminator” protocol, which involves cycling to 96° C. for 10 seconds, 50° C. for 5 seconds then 60° C. for four minutes. After these experiments, the full sample volume (within ±100 nl) was recovered.

Another well according to the invention, which had a smaller diameter sample region was prepared and loaded with 88 nl of reactant liquid. The upper end of the well was sealed with self-adhesive film. This well was cycled to 96° C. for 10 seconds and 50° C. for 5 seconds through 25 cycles. After this cycling, 65 nl of liquid was recovered. For comparison purposes, 88 nl of liquid was loaded into the well and then immediately recovered (i.e. without any thermal cycling) and 66 nl of liquid was recovered. This experiment indicates that the loss in the 88 nl sample was largely due to incomplete sample recovery as opposed to losses due to evaporation from the samples during thermal cycling.

For comparison purposes, a commercially available prior art multi-well plate was tested by pipetting 500 nl of liquid into a well of the plate and exposing the plate to 25 cycles of thermal cycling, wherein each cycle involved holding the liquid at 96° C. for 10 seconds followed by holding the liquid at 50° C. for 5 seconds. The liquid was then recovered from the well. On average, it was possible to recover only 250 nl of liquid after the thermal cycling. Thus, approximately 50% of the liquid was lost during thermal cycling.

As will be apparent to those skilled in the art in the light of the foregoing disclosure, many alterations and modifications are possible in the practice of this invention without departing from the spirit or scope thereof. For example:

This invention is not limited to a liquid 16 which includes any particular selection of reactants, solvents, samples, or other components.

This invention may be practiced by selectively increasing thermal conductivities of portions of a well.

Accordingly, the scope of the invention is to be construed in accordance with the substance defined by the following claims.

What is claimed is:

1. A method for thermal cycling of a liquid sample, the method comprising:
placing a volume of the liquid sample into a well;
varying a temperature of the liquid sample according to a desired temperature-time profile; and,
while varying the temperature of the liquid sample, maintaining a temperature of one or more regions on an inner surface of the well at temperatures at least 1½° C. greater than the temperature of the liquid sample, the one or more regions constituting 50 percent or more of an area of the inner surface of the well above a separation level.

2. The method of claim 1, wherein the separation level is one of:
a level of a top surface of the liquid sample;
a level separating an upper 50% of a volume of the well from a lower 50% of the volume of the well; and,
a level separating a lowermost 3 μl volume within the well from a remainder of a volume within the well.

3. The method of claim 2 wherein varying the temperature of the liquid sample comprises cycling the liquid sample between two or more temperatures, each of the two or more temperatures in the range of 40° C. to 100° C.

4. The method of claim 3 wherein the two or more temperatures include a first temperature in the range of 50° C. to 56° C.

5. The method of claim 3 wherein the two or more temperatures include a second temperature is in the range of 93° C. to 97° C.

6. The method of claim 1 wherein the volume of the liquid sample is less than 3 μl.

7. The method of claim 1 wherein the volume of the liquid sample is less than 1 μl.

8. The method of claim 1 wherein the one or more regions constitute 75 percent or more of the area of the inner surface of the well above the separation level.

9. The method of claim 8 wherein the volume of the liquid sample is less than 3 μl.

10. The method of claim 8 wherein the volume of the liquid sample is less than 1 μl.

11. The method of claim 1 wherein varying the temperature of the liquid sample comprises placing the well in thermal contact with a temperature-controlled block and varying a temperature of the temperature-controlled block.

12. The method of claim 11 wherein maintaining a temperature of one or more regions on an inner surface of the well at temperatures at least 1½° C. greater than the temperature of the liquid sample comprises placing the regions in thermal contact with a temperature-controlled plate and controlling a temperature of the temperature-controlled plate.

13. The method of claim 11 wherein maintaining a temperature of one or more regions on an inner surface of the well at temperatures at least 1½° C. greater than the temperature of the liquid sample comprises placing the regions in thermal contact with a gas or liquid having a temperature at least 1½° C. greater than the block.

14. The method of claim 1 comprising, prior to varying a temperature of the liquid sample according to a desired temperature-time profile, sealing the well by inserting a plug into an upper end of the well, the plug extending into a bore of the well.

15. The method of claim 1 wherein an interior of the well comprises a lowermost sample-holding portion having a first cross sectional area and an upper portion having a second cross sectional area greater than the first cross sectional area, wherein the volume of the liquid sample does not exceed a volume of the sample-holding portion.

16. A well for use in conjunction with a thermal cycling apparatus having a heated lid and a temperature-controlled block having a socket for receiving the well to expose a volume of a liquid to thermal cycling, the well comprising:
a wall having an inner surface surrounding a bore;
a sample-holding volume located in the bore at a lower end of the well;
wherein, there are one or more regions constituting 50% or more of an area of the inner surface of the well above the sample-holding volume, which regions, when the well is received in the socket, have relative thermal proximities of 1/19 or greater to the heated lid and relative thermal proximities of 19 or less to the block.

17. The well of claim 16 comprising a region of reduced thermal conductivity between the one or more regions and the lower end of the well.

18. The well of claim 17 wherein the region of reduced thermal conductivity extends circumferentially around the wall of the well.

19. The well of claim 18 wherein the region of reduced thermal conductivity comprises a region within which a thickness of the wall is reduced.

20. The well of claim 18 wherein the region of reduced thermal conductivity comprises a region within which the wall is made of a material having a reduced thermal conductivity in comparison to a material of the wall adjacent the sample-holding volume.

21. The well of claim 16 wherein the sample-holding volume has a volume of less than 3 μl.

22. The well of claim 16 wherein the sample-holding volume has a volume of less than 1 μl.

23. The well of claim 16 wherein the wall comprises a material having an increased thermal conductivity in its portions between the one or more regions and a portion of the well which engage the heated lid when the well is received in the socket.

24. The well of claim 23 wherein the material having an increased thermal conductivity comprises a later of a metal.

25. The well of claim 16 wherein, for points P₁ below the separation line, the following relationship holds:
$$\frac{1}{1 + \frac{{K}_{A}}{{K}_{D}}} < \frac{1}{50}$$
where K_{A} is the thermal contact between each point P₁ and the block and K_{D} is the thermal contact between each point P₁ and the lid.

26. The well of claim 25 wherein, for points P₂ in the one or more regions, the following relationship holds:
$$2 \frac{1}{2} < \frac{50}{1 + \frac{{K}_{C}}{{K}_{B}}}$$
where K_{C} is the thermal contact between point P₂ and the block and K_{B} is the thermal contact between point P₂ and the lid.

27. The well of claim 16 wherein, for points P₂ in the one or more regions, the following relationship holds:
$$2 \frac{1}{2} < \frac{50}{1 + \frac{{K}_{C}}{{K}_{B}}}$$
where K_{C} is the thermal contact between point P₂ and the block and K_{B} is the thermal contact between point P₂ and the lid.

28. The well of claim 16 wherein the sample-holding volume has a cross-sectional area smaller than a cross-sectional area of a bore of the well above the sample-holding volume.

29. A plate comprising an array of wells as claimed in claim 25.

30. A plate comprising an array of wells as claimed in claim 26.

31. A plate comprising an array of wells as claimed in claim 16.

32. Apparatus for performing thermal cycling on a volume of a liquid, the apparatus comprising:
a well comprising a wall having an inner surface surrounding a bore and a sample holding volume located in the bore at a lower end of the well;
a block comprising a socket for receiving the well and a temperature controller for controlling a temperature of the block; and,
a heated lid capable of being brought into good thermal contact with an upper end of the well;
wherein, when the well is received in the socket, the sample-holding volume has a first thermal contact with the block and one or more regions on the inner surface, which constitute 50 percent or more of an area of the inner surface of the well above the sample-holding volume, have a second thermal contact with the block, the first thermal contact being closer than the second thermal contact.

33. The apparatus of claim 32 wherein, when the well is received in the socket, the lower end of the well is touching the block and there is an air gap between the well and portions of the block above the sample-holding region.

34. The apparatus of claim 32 wherein an upper portion of the well comprises a layer of a material which is thermally insulating relative to a material of the lower end of the well.

35. The apparatus of claim 32 wherein the well comprises a region of reduced thermal conductivity between the one or more regions and the lower end of the well.

36. The apparatus of claim 35 wherein the region of reduced thermal conductivity extends circumferentially around the wall of the well.

37. The apparatus of claim 35 wherein the region of reduced thermal conductivity comprises a region within which a thickness of the wall is reduced.

38. The apparatus of claim 35 wherein the region of reduced thermal conductivity comprises a region within which the wall is made of a material having a reduced thermal conductivity in comparison to a material of the wall adjacent the sample-holding volume.

39. The apparatus of claim 35 wherein the sample-holding volume has a cross-sectional area smaller than a cross-sectional area of the bore above the sample-holding volume.

40. The apparatus of claim 32 wherein the one or more regions have relative thermal proximities to the block relative to the heated lid of 19 or less.

41. The apparatus of claim 40 wherein the one or more regions have thermal proximities to the heated lid relative to the block of 1/19 or greater.

42. The apparatus of claim 32 wherein the one or more regions have percentage thermal proximities to the block of 80% or less.

43. The apparatus of claim 40 wherein the one or more regions have percentage thermal proximities to the heated lid of 20% or greater.

44. The apparatus of claim 32 wherein the block comprises an array of sockets and the apparatus comprises a plurality of wells connected together and engageable in corresponding ones of the sockets.

45. The apparatus of claim 32 wherein the sample-holding volume has a volume of less than 3 μl.

46. The apparatus of claim 32 wherein the sample-holding volume has a volume of less than 1 μl.

47. The apparatus of claim 32 comprising a sealing member, the sealing member comprising a plug projecting downwardly into a bore of the well.

48. The apparatus of claim 47 wherein the plug has a truncated conical form.

49. The apparatus of claim 47 wherein the plug has a cylindrical form.

50. The apparatus of claim 47 comprising an o-ring seal on the plug, the o-ring seal sealingly engageable with the inner surface of the wall of the well.

51. An apparatus for performing thermal cycling on a liquid sample, the apparatus comprising:
a well comprising a wall surrounding a bore;
a block comprising a socket for receiving the well and a temperature controller for controlling the temperature of the block;
a heated lid capable of being brought into good thermal contact with an upper end of the well;
wherein when the well is received in the socket, the well comprises a lower region, which has a first thermal contact with the block, and an upper region comprising one or more portions that constitute 50% or more of an area of an inner surface of the wall, which have a second thermal contact with the block, the first thermal contact being closer than the second thermal contact.

52. The apparatus of claim 51 wherein points on the inner surface in the one or more portions of the upper region have percentage thermal proximities to the block of 80% or less.

53. The apparatus of claim 49 wherein points on the inner surface in the lower region have percentage thermal proximities to the block of 95% or more.

54. The apparatus of claim 51 wherein points on the inner surface in the one or more portions of the upper region have relative thermal proximities to the block relative to the heated lid of 19 or less.

55. The apparatus of claim 49 wherein points on the inner surface in the lower region have relative thermal proximities to the block relative to the heated lid of 1/19 or more.

56. The apparatus of claim 51, wherein a separation level between the lower region and the upper region is one of:
a level of a top surface of the liquid sample;
a level separating an upper 50% of a volume of the well from a lower 50% of the volume of the well; and,
a level separating a lowermost 3 μl volume within the well from a remainder of a volume within the well.

57. A well for use in conjunction with a thermal cycling apparatus having a heated lid and a temperature-controlled block having a socket for receiving the well to expose a volume of a liquid to thermal cycling, the well comprising:
a wall having an inner surface surrounding a bore;
the bore comprising a sample-holding volume located at a lower end of the bore, the sample holding volume having a first cross sectional area and a volume of 3 μl or less;
the bore comprising an upper portion having a second cross sectional area greater than the cross sectional area of the sample holding volume.

58. The well of claim 57 wherein the sample-holding volume comprises a portion of the bore having a circular cross section.

59. The well of claim 58 wherein the circular cross section of the sample holding volume has a constant diameter throughout at least 90% of the sample holding volume.

60. The well of claim 59 wherein the upper portion and sample holding volume are separated by a step in the bore.
